(19) United States
(12) Patent Application Publication
(10) Pub. No.: US 2009/0098071 A1
(43) Pub. Date: Apr. 16, 2009

(54) Cosmetic and dermatological light protection formulations in the form of O/W macroemulsions or O/W microemulsions, with a content of dihydroxyacetone

(75) Inventors: Albrecht Dorschner, Hamburg (DE); Anja Knuppel, Hamburg (DE)

Correspondence Address: GREENBLUM & BERNSTEIN, P.L.C., 1950 ROLAND CLARKE PLACE, RESTON, VA, 20191, US

(73) Assignee: Beiersdorf AG, Hamburg (DE)

(21) Appl. No.: 12/285,200

(22) Filed: Sep. 30, 2008

Related U.S. Application Data
(63) Continuation of application No. 11/106,257, filed on Apr. 14, 2005, now abandoned.
Application No. 11/106,257 is a division of application No. 09/678,766, filed on Oct. 2, 2000, now abandoned.

(30) Foreign Application Priority Data
Oct. 15, 1999 (DE) 199 49 826.1

(51) Int. Cl. A61K 8/35; A61Q 17/04

(52) U.S. Cl. 424/59

(57) ABSTRACT

A process for preparing a sprayable self-tanning oil-in-water microemulsion having an oil droplet diameter in a range of from about 10⁻¹ μm to about 10⁻² μm. The process comprises changing a pH of a mixture comprising an aqueous phase, an oil phase, from 0.5% to 6% by weight of dihydroxyacetone and from 0.1% to 5% by weight of one or more emulsifiers A whose lipophilicity is pH-dependent to effect a phase inversion which results in the microemulsion.

### CROSS-REFERENCE TO RELATED APPLICATIONS

The present application is a continuation of U.S. application Ser. No. 11/106,257, filed Apr. 14, 2005, the entire disclosure whereof is incorporated by reference herein, which is a divisional of U.S. application Ser. No. 09/678,766, filed Oct. 2, 2000, abandoned, which claims priority under 35 U.S.C. § 119 of German Patent Application No. 199 49 826.1, filed Oct. 15, 1999.

The present invention relates to cosmetic and dermatological preparations for tanning the skin, in particular to those which also offer protection against UV radiation.

The harmful effect of the ultraviolet part of solar radiation on the skin is generally known. While rays having a wavelength of less than 290 nm (the UVC region), are absorbed by the ozone layer in the earth's atmosphere, rays in the range between 290 nm and 320 nm, the UVB region, cause erythema, simple sunburn or even burns of varying severity.

The erythema activity maximum of sunlight is given as the relatively narrow region around 308 nm.

Numerous compounds are known for protecting against UVB radiation; these are mostly derivatives of 3-benzylidenecamphor, of 4-aminobenzoic acid, of cinnamic acid, of salicylic acid, of benzophenone and also of 2-phenylbenzimidazole.

It is also important to have available filter substances for the range between about 320 nm and about 400 nm, the UVA region, since its rays can also cause damage. Thus, it has been found that UVA radiation leads to damage of the elastic and collagenous fibers of connective tissue, causing premature aging of the skin, and that it is to be regarded as a cause of numerous phototoxic and photoallergic reactions. The harmful effect of UVB radiation can be intensified by UVA radiation.

In addition, UVA radiation can cause skin damage by damaging keratin or elastin in the skin. This leads to a reduction in elasticity and water-storage capacity, i.e. the skin becomes less supple and tends towards wrinkling. The notably high incidence of skin cancer in regions where solar irradiation is strong indicates that damage to the genetic information in cells is also apparently caused by sunlight, specifically by UVA radiation.

However, UV radiation can also lead to photochemical reactions, the photochemical reaction products interfering with the skin's metabolism.

Such photochemical reaction products are predominantly free-radical compounds, e.g. hydroxyl radicals. Undefined free-radical photo products which are formed in the skin itself can also display uncontrolled secondary reactions as a result of their high reactivity. However, singlet oxygen, a non-free-radical excited state of the oxygen molecule, can also arise during UV irradiation, as can short-lived epoxides and many others. Singlet oxygen, for example, differs from the normal triplet oxygen (free-radical ground state) by virtue of its increased reactivity. However, excited, reactive (free-radical) triplet states of the oxygen molecule also exist.

UV radiation is also a type of ionizing radiation. There is therefore the risk that ionic species may also arise during UV exposure, which then, for their part, are capable of oxidative intervention in the biochemical processes.

The pigmentation of human skin is essentially brought about by the presence of melanin. Melanin and its degradation products (melanoids), carotene, degree of perfusion, and the condition and thickness of the Stratum corneum and other skin layers permit skin shades from virtually white (in cases of reduced filling or in cases of an absence of blood vessels) or yellowish via pale brown-reddish, bluish to brown of different shades and finally almost black. The individual regions of the skin display differing depths of shade as a result of varying amounts of melanin.

Natural melanin protects the skin from penetrating UV radiation. The number of melanin granules produced in the melanocytes determines whether a person has pale skin or dark skin. In cases of strong pigmentation (e.g. in colored races, but also in those with pale skin following UV irradiation) melanin is also to be found in the Stratum spinosum and even in the Stratum corneum. It attenuates the UV radiation by up to about 90% before it reaches the corium.

Depending on their sensitivity to light, the skin types below are normally differentiated:

| | |
|---|---|
| Skin type I | never tans, always burns. |
| Skin type II | rarely tans, burns easily. |
| Skin type III | tans averagely well. |
| Skin type IV | tans easily to give a lasting tan, almost never burns. |
| Skin type V | dark, often almost black skin, never burns. |

The natural shielding from harmful UV radiation is a tangible advantage of natural skin tanning. Moreover, for many decades a “healthy” skin color has been a sign of, in particular, sporting activity and is therefore considered to be desirable by a broad class of consumer. Representatives of skin types I and II who wish to enjoy such a skin shade in any case therefore have to rely on self-tanning preparations. However, representatives of skin type III who do not wish to excessively be exposed to the risks of sunbathing but nevertheless want to appear tanned are also thankful target groups for self-tanning preparations.

Artificial skin tanning can be brought about in a cosmetic or medicinal way, the following approaches essentially playing a role:

The regular taking of carotene preparations results in carotene being stored in the subcutaneous fatty tissue, and the skin gradually turns orange to yellow-brown.

Using make-up preparations which can be washed off it is possible to achieve a slight skin shading (e.g. extracts of fresh green walnut shells, henna).

Coloring can also take place via the route of a chemical change in the horny layer of the skin using self-tanning preparations. The most important active ingredient is dihydroxyacetone (DHA). The skin tanning achieved in this way cannot be washed off and is removed only with the normal flaking of the skin (after about 10-15 days).

### Dihydroxyacetone is Characterized by the Structure

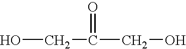

Dihydroxyacetone (1,3-dihydroxypropan-2-one) is a colorless solid with a characteristic odor. In freshly prepared aqueous solutions it is present as dimer which segments into the monomers by heating. It can be referred to as ketotriose and reacts as a reducing sugar with the amino acids of the skin and the free amino and imino groups of the keratin via a number of intermediates in the sense of a Maillard reaction to give brown-colored substances, so-called melanoids, which are sometimes also called melanoidins.

A disadvantage of tanning with dihydroxyacetone is that the skin tanned therewith is not protected from sunburn, in contrast to “sun-tanned” skin.

A further disadvantage of dihydroxyacetone is that, particularly under the influence of ultraviolet radiation, formaldehyde is eliminated, albeit in small amounts in most cases. There was therefore an urgent need to find ways in which the decomposition of dihydroxyacetone can be effectively countered.

To overcome the disadvantages of the prior art was therefore the object of the present invention.

Customary cosmetic forms of application are emulsions. This term generally means a heterogeneous system of two liquids which are immiscible or miscible only to a limited extent with one another, which are usually referred to as phases. One is in the form of droplets (disperse or internal phase), while the other liquid forms a continuous (coherent or internal) phase. Less common forms of application are multiple emulsions, i.e. those which, in the droplets of the dispersed (or discontinuous) phase, comprise for their part droplets of a further dispersed phase, e.g. W/O/W emulsions and O/W/O emulsions.

More recent findings have recently led to a better understanding of cosmetic emulsions which are of relevance in practice. Here, it is assumed that the emulsifier mixtures used in excess form lamellar liquid-crystalline phases or crystalline gel phases. In the gel network theory, stability and physicochemical properties of such emulsions are attributed to the formation of viscoelastic gel networks.

If the two liquids are water and oil, and oil droplets are finely dispersed in water, an oil-in-water emulsion (O/W emulsion, e.g. milk) results. The basic character of an O/W emulsion is defined by the water. In the case of a water-in-oil emulsion (W/O emulsion, e.g. butter) the principle is reversed, the basic character being determined here by the oil.

In order to be able to ensure the metastability of emulsions, interface-active substances, i.e. emulsifiers, are usually necessary. The use of customary cosmetic emulsifiers is in itself entirely acceptable. Nevertheless, emulsifiers, as ultimately any chemical substance, can in isolated cases cause allergic reaction or reactions based on oversensitivity of the user. For example, it is known that in some particularly sensitive people, certain light dermatoses are triggered by certain emulsifiers and simultaneous action of sunlight.

It is possible to prepare emulsifier-free preparations which, for example, have, in an aqueous phase, dispersed oil droplets, similar to an O/W emulsion. A prerequisite for this may be that the continuous aqueous phase has a gel framework which stabilizes the dispersed phase, and other conditions besides. Such systems are sometimes called hydrodispersions or oleodispersions depending on which is the disperse phase and which is the continuous phase.

However, for cosmetic technology it is neither necessary nor possible to dispense with emulsifiers completely, not least because there is a certain choice of particularly mild emulsifiers. However, the prior art lacks a satisfactorily broad variety of such emulsifiers which then would also significantly broaden the application spectrum of correspondingly mild cosmetic preparations which are tolerated by the skin.

Thus, a further object of the present invention was to provide cosmetic and dermatological preparations having excellent skincare properties.

The person skilled in the art is naturally aware of a large number of ways of formulating stable O/W preparations for cosmetic or dermatological use, for example in the form of creams and ointments, which are spreadable in the range from room temperature to skin temperature, or as lotions and milks, which are more likely flowable in this temperature range. However, there are only a few formulations in the prior art which are of sufficiently low viscosity that they would, for example, be sprayable.

In addition, low-viscosity preparations of the prior art frequently have the disadvantage that they are unstable, and are limited to a narrow field of application or to a limited choice of feed material. Low-viscosity products in which, for example, strong polar oils—such as the vegetable oils otherwise frequently used in commercially available products—are sufficiently stabilized are therefore currently not on the market.

The term ‘viscosity’ means the property of a liquid to resist the mutual laminar displacement of two neighboring layers (internal friction). This so-called dynamic viscosity is nowadays defined according to η=t/D as the ratio of shear stress to the velocity gradient perpendicular to the direction of flow. For Newtonian liquids, η is a material constant having the SI unit Pascal second (Pa·s) at a given temperature.

The quotient ν=η/ρ from the dynamic viscosity η and the density ρ of the liquid is referred to as the kinematic viscosity ν and is given in the SI unit m²/s.

Fluidity (φ) is the inverse of viscosity (φ=1/η). In the case of ointments and the like, the use value is inter alia determined by the so-called tack. The tack of an ointment or ointment base or the like means its property to draw threads of varying lengths when a small sample is removed; accordingly, a distinction is made between short- and long-stretch substances.

While the graphical representation of the flow behavior of Newtonian liquids at a given temperature produces a straight line, in the case of so-called non-Newtonian liquids considerable deviations often arise, depending on the particular velocity gradient D (shear rate \dot{γ}) or the shear stress τ. In these cases, the so-called apparent viscosity can be determined which, although not bound to the Newtonian equation, can be used to determine the true viscosity values by graphical methods.

Falling-body viscometry is suitable only for investigating Newtonian liquids and gases. It is based on Stokes' law, according to which, for the falling of a sphere through a liquid which flows around it, the dynamic viscosity η can be determined from

$$η = \frac{2 {r}^{2} ( {ρ}_{k} - {ρ}_{F 1} ) \cdot g}{9 \cdot v}$$

where
r=radius of the sphere, v=fall velocity, ρ_{K}=density of the sphere, η_{FI}=density of the liquid and g=acceleration of the fall.

O/W emulsions with a low viscosity which have a storage stability as is required for marketable products can only be formulated in accordance with the prior art in a very complex manner. Accordingly, the supply of such formulations is extremely low. Nevertheless, formulations of this type could offer the consumer hitherto unknown cosmetic results.

An object of the present invention was to make available preparations which have a very low viscosity and do not have the disadvantages of the prior art.

For polyol fatty acid esters, the definition of the HLB value is given by the formula I

HLB=20*(1−S/A)

For a group of emulsifiers whose hydrophilic moiety consists only of ethylene oxide units, the formula II applies

HLB=E/5

where
S=saponification number of the ester,
A=acid number of the recovered acid
E=mass fraction of ethylene oxide (in %) based on the overall molecule.

Emulsifiers with HLB values of 6-8 are generally W/O emulsifiers, and those with HLB values of 8-18 are generally O/W emulsifiers.

Literature: “Kosmetik—Entwicklung, Herstellung und Anwendung kosmetischer Mittel [Cosmetics—Development, Preparation and Use of Cosmetic Compositions]”; W. Umbach (Ed.), Georg Thieme Veriag 1988.

Hydrophilic emulsifiers (with high HLB values) are generally O/W emulsifiers. Accordingly, hydrophobic or lipophilic emulsifiers (with low HLB values) are generally W/O emulsifiers.

U.S. Pat. No. 4,931,210 describes a process for the preparation of W/O/W emulsions where polyglycerol polyricinoleates are used as emulsifiers.

The droplet diameters of customary “simple”, i.e. non-multiple emulsions are in the range from about 1 μm to about 50 μm. Such “macroemulsions” are, without further coloring additives, milky-white in color and opaque. Finer “macroemulsions”, the droplet diameters of which are in the range from about 10⁻¹ μm to about 1 μm are, again without coloring additives, bluish-white in color and opaque. Such “macroemulsions” usually have high viscosity.

Only micellar and molecular solutions having particle diameters of less than about 10⁻² μm, but which are no longer to be regarded as true emulsions, may have a clear and transparent appearance.

By contrast, the droplet diameter of microemulsions is in the range from about 10⁻² μm to about 10⁻¹ μm. Microemulsions are translucent and in most cases of low viscosity. The viscosity of many microemulsions of the O/W type is comparable with that of water.

The advantage of microemulsions is that, in the disperse phase, active ingredients can be present in considerably more finely disperse form than in the disperse phase of “macroemulsions”. A further advantage is that they are sprayable as a result of their low viscosity. If microemulsions are used as cosmetics, corresponding products are characterized by high cosmetic elegance.

It is known that hydrophilic emulsifiers change their solubility behavior from water-soluble to fat-soluble with increasing temperature. The temperature range in which the emulsifiers have changed their solubility is called the phase inversion temperature range (PIT).

T. J. Lin, H. Kurihara and H. Ohta (Journal of the Society of Cosmetic Chemists 26, pp. 121-139, March 1975) show that for nonpolar oils extremely unstable multiple emulsions may be present in the PIT range.

The object of the present invention was therefore to remedy these shortcomings.

Surprisingly, we have found, and herein lies the basis of the achievement of the objects, that oil-in-water emulsions, in particular O/W microemulsions
(a) comprising at least one emulsifier (emulsifier A), chosen from the group of emulsifiers having the following properties
   their lipophilicity is either dependent on the pH inasmuch as an increase or decrease in the pH results in an increase or decrease in lipophilicity, it being unimportant which of the two possibilities for change in the lipophilicity is effected by the increase or decrease in pH, and/or
   their lipophilicity is dependent on the temperature inasmuch as the lipophilicity increases with increasing temperature and their hydrophilicity increases with decreasing temperature,
(b) also optionally further substances which are soluble or dispersible in the oil phase or the water phase, preferably including those chosen from the group of emulsifiers not covered by the definition of emulsifier A, in particular those which act primarily as W/O emulsifiers,
(c) an effective amount of dihydroxyacetone,
   overcome the disadvantages of the prior art.

Within the meaning of the present invention, if the phase inversion is essentially initiated by varying the temperature, O/W emulsions, in particular O/W microemulsions are obtainable, where by the size of the oil droplets is essentially determined by the concentration of the emulsifier(s) used inasmuch as a higher emulsifier concentration brings about smaller droplets, and a lower emulsifier concentration leads to relatively large droplets. If phase inversion is essentially triggered by varying the temperature, it is entirely advantageous to dispense with further emulsifiers not covered by the definition of the emulsifier A, namely W/O emulsifiers.

The total amount of dihydroxyacetone in the finished cosmetic or dermatological preparations is advantageously chosen from the range 0.1-10.0% by weight, preferably 0.5-6.0% by weight, based on the total weight of the preparations.

If phase inversion is essentially triggered by varying the pH, O/W emulsions, in particular O/W microemulsions are obtainable. If phase inversion is essentially triggered by varying the pH, it is entirely advantageous to use one or more further emulsifiers not covered by the definition of the emulsifier A, namely W/O emulsifiers.

According to the invention it is possible to obtain O/W microemulsions if the oil phase proportion is below about 20% by weight, in particular below about 15% by weight, based on the total weight of the preparation, if less than about 5% by weight of an additional W/O emulsifier not covered by the definition of the emulsifier A are present and/or if the oil phase has a high content of polar oils.

According to the invention, O/W emulsions (“macroemulsions”) can be obtained if less than about 5% by weight of an additional W/O emulsifier not covered by the definition of the emulsifier A, and more than about 20% by weight of a polar oil phase are present. Advantageously additional gel formers (e.g. Carbopols, xanthan gum, cellulose derivatives) can be used.

In individual cases it is possible to slightly exceed or fall below the abovementioned concentration limits and nevertheless obtain the emulsion types in question. In view of the wide-ranging diversity of suitable emulsifiers and oil constituents, this is not entirely unexpected for the person skilled in the art in that he knows that such excesses or deficits do not depart from the basis of the present invention.

If phase inversion is essentially triggered by varying the temperature, O/w emulsions, in particular O/W microemulsions, are obtainable, whereby the size of the oil droplets is essentially determined by the concentration of the emulsifier(s) used, inasmuch as a higher emulsifier concentration brings about smaller droplets and a lower emulsifier concentration leads to relatively large droplets. If phase inversion is essentially triggered by varying the temperature, it is entirely advantageous, although not obligatory, to dispense with further emulsifiers not covered by the definition of the emulsifier A, namely W/O emulsifiers.

If phase inversion is essentially triggered by varying the pH, O/W emulsions, in particular O/W microemulsions, but also O/W/O emulsions, are obtainable. If phase inversion is triggered essentially by varying the pH, it is entirely advantageous to use one or more further emulsifiers not covered by the definition of the emulsifier A, namely W/O emulsifiers.

In individual cases it is possible to slightly exceed or fall below the abovementioned concentration limits and nevertheless obtain the emulsion types in question. In view of the wide-ranging diversity of suitable emulsifiers and oil constituents, this is not entirely unexpected for the person skilled in the art in that he knows that such excesses or deficits do not depart from the basis of the present invention.

Surprisingly, we have found that the pigment particle(s) used according to the invention are in the form of solids, and to a certain extent “encapsulated”, namely separate from other constituents of the preparations, in some of which they can even have limited solubility. It is assumed that the solid particles of the sparingly soluble UV filter substances receive a coating film as a result of the incorporation process according to the invention, which film presumably comprises emulsifier molecules as an essential constituent.

According to the invention the recrystallization of the s-triazine derivative(s) used according to the invention can be prevented. Moreover, light protection preparations are obtainable according to the invention which have excellent use properties.

### BRIEF DESCRIPTION OF THE DRAWINGS

FIG. 1 is an emulsion phase diagram wherein a concentration parameter P is plotted against the temperature θ.

FIG. 2 is another emulsion phase diagram wherein a concentration parameter P is plotted against the temperature θ.

FIG. 3 is an emulsion phase diagram wherein the concentration of the water phase is plotted against the temperature θ.

FIG. 4 is an emulsion phase diagram wherein the concentration of emulsifier(s) is plotted against the temperature θ.

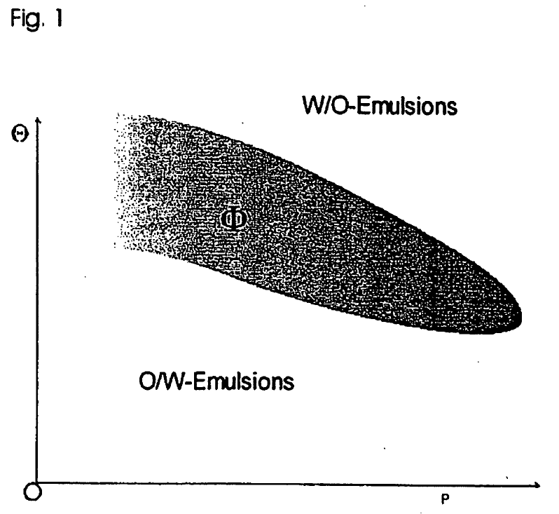

FIG. 1 shows a very simplified representation of a phase diagram. The variable parameter P is plotted against the temperature θ as a second variable. P is here a concentration parameter, either the proportion of the oil phase, the proportion of the water phase or the concentration of an emulsifier or an emulsifier mixture. For systems according to the invention it is the case that at relatively low temperatures an O/W emulsion is present and as the temperature increases the phase inversion range can be passed through. If the temperature is increased further, W/O emulsions are observed. The structure of the system in the phase inversion range is seemingly unimportant for the present invention. For example, it is conceivable that lamellar phases, bicontinuous phases, cubic, hexagonal or inverse hexagonal phases are present in the phase inversion range, and also that the phase inversion range is composed of two or more identical or more or less different phases.

The phase inversion range can be represented mathematically as a point quantity within the straight-line coordinate system Σ, which is formed by the parameters of temperature, the concentration of a suitable emulsifier or of an emulsifier mixture in the preparation and the respective concentrations of the oil phase and water phase, according to:

Σ={O, θ, m, H, W},

where
O— coordinate origin
θ—temperature
m—concentration of the emulsifier/emulsifier mixture
H—concentration of the oil phase
W—concentration of the water phase

Strictly speaking of course, in a multicomponent emulsifier system, the contribution mᵢ of each individual emulsifier to the overall function must be taken into consideration, which, in the case of an i-component emulsifier system, leads to the relationship

Σ={O, θ, m₁, m₂, . . . , mᵢ, H, W}.

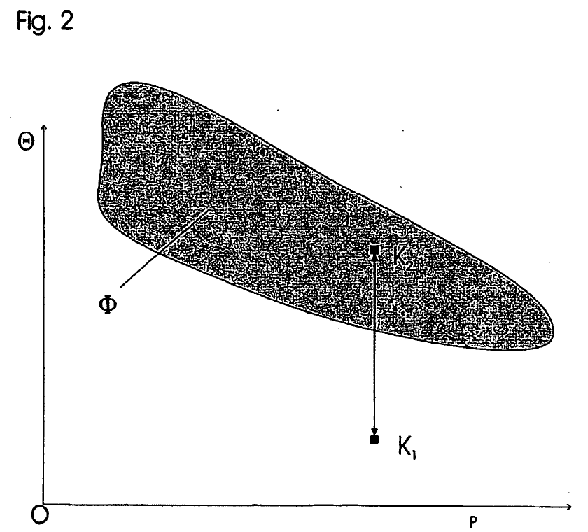

The phase inversion range Φ here in the mathematical sense is a continuous region or a large number of continuous regions within the coordinate system Σ. Φ represents the total amount of coordinate points K(θ, a, m₁, m₂, . . . mᵢ, H, W), which determine mixtures according to the invention of a water phase of concentration W, oil phase of concentration H, i emulsifiers according to the invention of concentration mᵢ at the temperature θ, and for which, upon passing from a coordinate K₁∉Φ to a coordinate K₂∈Φ, phase inversion occurs, as described in FIG. 2.

It is irrelevant here whether the phase inversion range of a given system is a single continuous (i+3)-dimensional field or consists of two or more such fields which are continuous but separate from one another, i.e. corresponding to two or more phase inversion ranges of a given system. Within the scope of the disclosure presented herein, “the” or “a” phase inversion range is always referred to in general terms, even if two or more such ranges separate from one another are present.

The variable coordinates given in FIG. 2 are temperature 0 and the above-described concentration parameter P, it being possible for it to remain open which specific concentration parameter is involved. On passing from K₁ to K₂, only the temperature is increased; and the other variables are kept constant.

Under the conditions according to the invention, this process is not reversible, i.e. if the system reverts from the coordinate K₂∈Φ to coordinate K₁∉Φ, transparent O/W microemulsions may be obtained according to the invention.

The practice of preparing a microemulsion according to the invention accordingly advantageously consists, after choosing suitable raw materials, i.e. water phase and oil phase, one or more O/W emulsifiers used according to the invention, the latter being present in concentrations at which phase inversion is possible for the given mixture, and optionally further substances, in combining the individual components with stirring, bringing about a phase inversion by increasing the temperature of the mixture, and thereafter allowing the mixture to cool to room temperature with continued stirring.

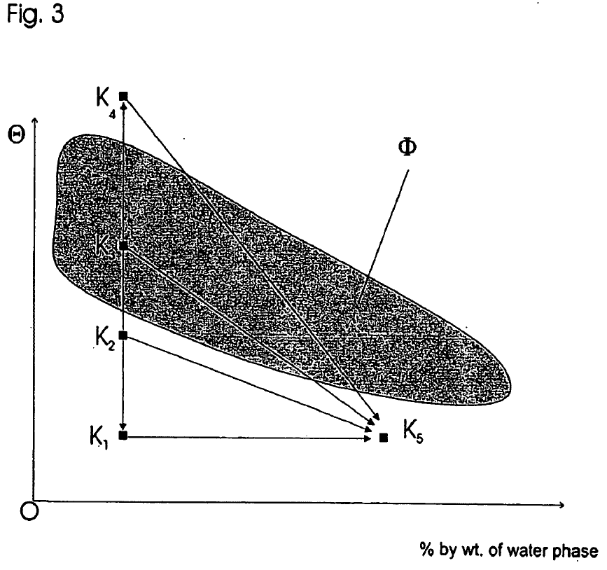

However, it is also possible here to vary two or more parameters at the same time, as shown in FIG. 3. In FIG. 3 the concentration of the water phase is plotted against the temperature. Starting from the coordinate K₁∉Φ, by increasing the temperature, while maintaining all other parameters, the coordinates K₂∉Φ and K₄∉Φ can be reached, or K₃∈Φ. Starting from the coordinates K₃ and K₄, by lowering the temperature, while maintaining all other parameters, back to coordinate K₁, O/W microemulsions according to the invention can be obtained.

Starting from the coordinates K₃ and K₄, by lowering the temperature, and by additionally varying the concentration of the oil phase, in FIG. 3 by the addition of water, the coordinate K₅ can be reached and O/W microemulsions according to the invention can be obtained.

In view of FIG. 3, it is logical that starting from coordinate K₄, although this is outside the phase inversion range, systems similar to those which start from K₃ can be obtained, since starting from K₄, if the temperature is lowered, the phase inversion range must indeed also automatically be traversed.

Also, starting from the coordinate K₁, by varying the concentration of the water phase, i.e. for example by adding water, as is shown in FIG. 3, the coordinate K₅ is reached, and O/W microemulsions according to the invention can be obtained. In this regard, however, it must first be mentioned that in this case an O/W microemulsion, to a certain extent as a concentrate, must already be present, which is then converted into an O/W microemulsion according to the invention of different composition by dilution.

However, having said all that, it was surprising and therefore indicative of independent inventive activity, that starting from the coordinate K₂, which lies outside the phase inversion range, either by simply varying the temperature back to coordinate K₁ or by additionally varying the concentration of the oil phase, i.e., for example, by additional dilution with a water phase to coordinate K₅, O/W microemulsions according to the invention are also obtainable without passing through phase inversion. This is advantageously effected by bringing a mixture of the base components, comprising water phase, oil phase, one or more of the O/W emulsifiers used according to the invention, if desired one or more W/O emulsifiers, and optionally further auxiliaries, additives and/or active ingredients, which form an O/W emulsion below the phase inversion temperature range, to a temperature
at which the components which are soluble in the oil phase are present either in dissolved form or at least in the molten state
and which corresponds at least to the melting temperature of the highest-melting oily component which is not present in the dissolved state,
which is below the phase inversion temperature range of the system,
   and afterwards cooling the resulting O/W emulsion to room temperature to form an O/w microemulsion. This is preferably carried out with stirring.

This process according to the invention is particularly suitable if heat-sensitive or readily volatile substances are to be incorporated into the O/W microemulsions according to the invention. Moreover, this process, which is carried out at relatively low temperatures, is energy-saving compared with customary processes.

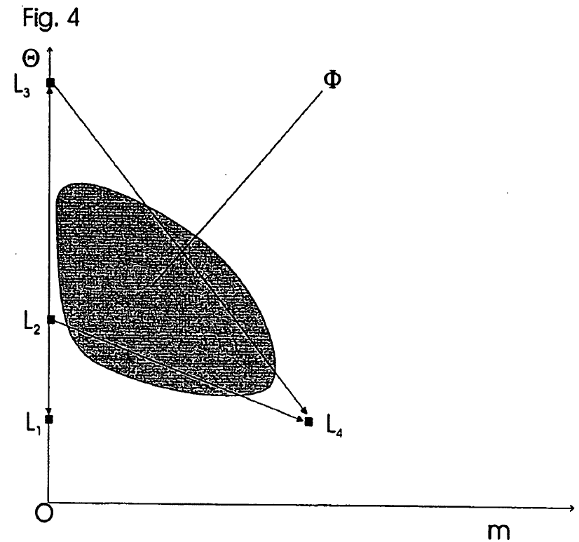

FIG. 4 describes the case in which no O/W emulsifier according to the invention is initially present in the coordinate L₁, and in which the system is brought to a coordinate L₃∉Φ or to a coordinate L₂∉Φ by increasing the temperature. The coordinate L₂ can of course also be achieved by cooling a system present in the coordinate L₃. The coordinates L₂ and L₃, in which, for example, W/O emulsions can be present, differ in principle merely by virtue of the fact that the temperature assigned to L₃ is higher than any temperature which can be assigned to the phase inversion temperature range.

The presence of an additional W/O emulsifier for systems which are symbolized in FIG. 4 is not necessarily required, but is advantageous. Addition of an O/W emulsifier according to the invention or of two or more such emulsifiers in the coordinates L₂ or L₃, on lowering the temperature, conveys the system to coordinate L₄, at which an O/W microemulsion according to the invention is then present.

A further advantageous embodiment of the process according to the invention accordingly consists, following the choice of suitable raw materials, i.e. water phase and oil phase and optionally further substances, in bringing the individual components, with stirring, to a temperature at which phase inversion is possible for the given mixture and, by adding the O/W emulsifier used according to the invention or the O/W emulsifiers used according to the invention to the mixture, bringing about phase inversion, and thereafter allowing the mixture to cool to room temperature with continued stirring.

It is not beyond the ability of the person skilled in the art to determine, by simple experiments, the suitable temperature range within which a given mixture can pass through phase inversion. This temperature range is usually to be chosen between 70 and 95° C., but in an individual case can also be above or below this.

In practice, it is possible and in some cases even advantageous for the temperature range which can be assigned to the phase inversion range to also be exceeded during the preparation of a microemulsion according to the invention since this range is then automatically traversed upon cooling to room temperature.

The practice of the preparation of an emulsion according to the invention advantageously consists, after choosing suitable raw materials, i.e. water phase and oil phase, one or more emulsifiers of type A, the latter being present in concentrations at which phase inversion is possible for the given mixture, and optionally further substances, in heating the individual components with stirring to a temperature at which phase inversion is possible for the given mixture, and, by increasing or decreasing the pH of the mixture, bringing about phase inversion, and afterwards allowing the mixture to cool to room temperature with continued stirring. One or more intermediate homogenization steps are advantageous, but are not absolutely necessary.

A further advantageous embodiment of the process according to the invention consists, after choosing suitable raw materials, i.e. water phase and oil phase, one or more emulsifiers of the type A, the latter being present in concentrations at which phase inversion is possible for the given mixture, and optionally further substances, in bringing the individual components, with stirring, to a pH at which phase inversion is possible for the given mixture, and, by increasing the temperature of the mixture, bringing about phase inversion, and afterwards allowing the mixture to cool to room temperature with continued stirring. One or more intermediate homogenization steps are advantageous, but are not absolutely necessary.

A third advantageous embodiment of the process according to the invention consists, after choosing suitable raw materials, i.e. water phase and oil phase, one or more emulsifiers of the type A and optionally further substances, in bringing the individual components, with stirring, to a pH and a temperature at which phase inversion is possible for the given mixture, and, by the addition of the emulsifier A or the emulsifiers A to the mixture, bringing about phase inversion, and afterwards allowing the mixture to cool to room temperature with continued stirring. One or more intermediate homogenization steps are advantageous, but are not absolutely necessary.

In practice, it is possible and in some cases even advantageous for the temperature range which can be assigned to the phase inversion range to also be exceeded during the preparation of an emulsion according to the invention since this range is then automatically traversed upon cooling to room temperature.

It is advantageous to buffer preparations according to the invention, preferably in the range of pH=4.5-5.5, in particular pH=5.

Cosmetic and dermatological preparations according to the invention comprise inorganic pigments, which are X-ray amorphous or non-X-ray amorphous, based on metal oxides and/or other metal compounds which are sparingly soluble or insoluble in water, in particular the oxides of titanium (TiO₂), zinc (ZnO), iron (e.g. Fe₂O₃, zirconium (ZrO₂), silicon (SiO₂), manganese (e.g. MnO), aluminum (Al₂O₃), cerium (e.g. Ce₂O₃), mixed oxides of the corresponding metals, and mixtures of such oxides. Particular preference is given to pigments based on TiO₂.

X-ray amorphous oxide pigments are metal oxides or semimetal oxides which reveal no or no recognizable crystal structure in X-ray diffraction experiments. Such pigments are often obtainable by flame reaction, for example by reacting a metal or semimetal halide with hydrogen and air (or pure oxygen) in a flame.

In cosmetic dermatological or pharmaceutical formulations, X-ray-amorphous oxide pigments are used as thickeners and thixotropic agents, flow auxiliaries, for emulsion and dispersion stabilization and as a carrier substance (for example for increasing the volume of finely divided powders).

X-Ray-amorphous oxide pigments which are known and are often used in cosmetic or dermatological-pharmaceutical are the silicon oxides of the Aerosil® grade (CAS No. 7631-86-9). Aerosils®, available from DEGUSSA, are characterized by low particle size (e.g. between 5 and 40 nm), where the particles are to be regarded as spherical particles of very uniform dimension. Macroscopically, Aerosils® are recognizable as loose, white powders. Within the meaning of the present invention, X-ray-amorphous silicon dioxide pigments are particularly advantageous and, of these, precisely those of the Aerosil® grade are preferred.

Advantageous Aerosil® grades are, for example, Aerosil® OX50, Aerosil® 130, Aerosil® 150, Aerosil® 200, Aerosil® 300, Aerosil® 380, Aerosil® MOX 80, Aerosil® MOX 170, Aerosil® COK 84, Aerosil® R 202, Aerosil® R 805, Aerosil® R 812, Aerosil® R 972, Aerosil® R 974, Aerosil® R976.

According to the invention, cosmetic or dermatological light protection preparations advantageously comprise 0.1 to 20% by weight, advantageously 0.5 to 10% by weight, very particularly preferably 1 to 5% by weight, of X-ray-amorphous oxide pigments.

According to the invention, the non-X-ray-amorphous inorganic pigments are advantageously present in hydrophobic form, i.e. they have been surface-treated to repel water. This surface treatment can involve providing the pigments with a thin hydrophobic layer by methods known per se.

Such a method consists, for example, in producing the hydrophobic surface layer according to a reaction as in

nTiO₂+m(RO)₃Si—R′->nTiO₂(surf.).

n and m are stoichiometric parameters to be used as desired, and R and R′ are the desired organic radicals. Hydrophobicized pigments prepared as in DE-A 33 14 742, for example, are advantageous.

Advantageous TiO₂ pigments are available, for example, under the tradenames T 805 from Degussa.

The total amount of inorganic pigments, in particular hydrophobic inorganic micropigments, in the finished cosmetic or dermatological preparations is advantageously chosen from the range 0.1-30% by weight, preferably 0.1-10.0% by weight, based on the total weight of the preparations.

The emulsifiers A are preferably chosen from the group of emulsifiers which are good proton donors or proton acceptors, it having to be ensured that their lipophilicity is either dependent oh the pH inasmuch as an increase or decrease in the pH results in an increase or decrease in lipophilicity, it being unimportant which of the two possibilities for change in the lipophilicity is effected by the increase or decrease of the pH, or their lipophilicity is dependent on the temperature inasmuch as the lipophilicity increases with increasing temperature and their hydrophilicity increases with decreasing temperature, or their lipophilicity is dependent on the pH and temperature inasmuch as an increase or decrease in the pH results in an increase or decrease in lipophilicity, it being unimportant which of the two possibilities for change in the lipophilicity is effected by the increase or decrease of the pH, and that the lipophilicity increases with increasing temperature and their hydrophilicity increases with decreasing temperature.

The emulsions according to the invention are advantageously notable for the fact that the emulsifier A or the emulsifiers A is or are present in concentrations of 0.01-20% by weight, preferably 0.05-10% by weight, particularly preferably 0.1-5% by weight, in each case based on the total weight of the composition.

The emulsifier(s) A is/are particularly advantageously chosen from the group of mono-, oligo- and polyethoxylated compounds, in particular polyethoxylated mono- or polybasic alcohols or fatty acids, for example
fatty alcohol ethoxylates
ethoxylated wool wax alcohols,
polyethylene glycol ethers of the general formula R—O—(—CH₂—CH₂—O—)ₙ—R′,
fatty acid ethoxylates of the general formula

R—COO—(—CH₂—CH₂—O—)ₙ—H.
etherified fatty acid ethoxylates of the general formula

R—COO—(—CH₂—CH₂—O—)ₙ—R′,
esterified fatty acid ethoxylates of the general formula

R—COO—(—CH₂—CH₂—O—)ₙ—C(O)R′.
polyethylene glycol glycerol fatty acid esters
ethoxylated sorbitan esters
cholesterol ethoxylates
ethoxylated triglycerides
alkyl ether carboxylic acids of the general formula R—O—(—CH₂—CH₂—O—)ₙ—CH₂—COOH and n is a number from 5 to 30,
polyoxyethylene sorbitol fatty acid esters,
alkyl ether sulfates of the general formula R—O—(—CH₂—CH₂—O—)ₙ—SO₃—H
fatty alcohol propoxylates of the general formula

R—O—(—CH₂—CH(CH₃)O—)ₙ—H,
polypropylene glycol ethers of the general formula

R—O—(—CH₂—CH(CH₃)O—)ₙ—R′,
propoxylated wool wax alcohols,
etherified fatty acid propoxylates

R—COO—(—CH₂—CH(CH₃)—O—)ₙ—R′,
esterified fatty acid propoxylates of the general formula

R—COO—(—CH₂—CH(CH₃)—O—)ₙ—C(O)—R′,
fatty acid propoxylates of the general formula

R—COO—(—CH₂—CH(CH₃)—O—)ₙ—H,
polypropylene glycol glycerol fatty acid esters
propoxylated sorbitan esters
cholesterol propoxylates
propoxylated triglycerides
alkyl ether carboxylic acids of the general formula

R—O—(—CH₂—CH(CH₃)O—)ₙ—CH₂—COOH
alkyl ether sulfates or the parent acids of these sulfates of the general formula

R—O—(—CH₂—CH(CH₃)O—)ₙ—SO₃—H
fatty alcohol ethoxylates/propoxylates of the general formula

R—O—Xₙ—Yₘ—H,
polypropylene glycol ethers of the general formula

R—O—Xₙ—Yₘ—R′,
etherified fatty acid propoxylates of the general formula

R—COO—Xₙ—Yₘ—R′,
fatty acid ethoxylates/propoxylates of the general formula

R—COO—Xₙ—Yₘ—H.

According to the invention, the emulsifiers A used are particularly advantageously chosen from the group of substances having HLB values of 11-18, very particularly advantageously having HLB values of 14.5-15.5, provided the emulsifiers A have saturated radicals R and R′. If the emulsifiers A have unsaturated radicals R and/or R′, or isoalkyl derivatives are present, then the preferred HLB value of such emulsifiers can also be lower or higher.

It is advantageous to choose the fatty alcohol ethoxylates from the group of ethoxylated stearyl alcohols, cetyl alcohols, cetylstearyl alcohols. Particular preference is given to:

polyethylene glycol(13) stearyl ether (steareth-13), polyethylene glycol(14) stearyl ether (steareth-14), polyethylene glycol(15) stearyl ether (steareth-15), polyethylene glycol(16) stearyl ether (steareth-16), polyethylene glycol(17) stearyl ether (steareth-17), polyethylene glycol(18) stearyl ether (steareth-18), polyethylene glycol(19) stearyl ether (steareth-19), polyethylene glycol(20) stearyl ether (steareth-20),
polyethylene glycol(12) isostearyl ether (isosteareth-12), polyethylene glycol(13) isostearyl ether (isosteareth-13), polyethylene glycol(14) isostearyl ether (isosteareth-14), polyethylene glycol(15) isostearyl ether (isosteareth-15), polyethylene glycol(16) isostearyl ether (iso-steareth-16), polyethylene glycol(17) isostearyl ether (isosteareth-17), polyethylene glycol (18) isostearyl ether (isosteareth-18), polyethylene glycol(19) isostearyl ether (iso-steareth-19), polyethylene glycol(20) isostearyl ether (isosteareth-20),
polyethylene glycol(13) cetyl ether (ceteth-13), polyethylene glycol(14) cetyl ether (ceteth-14), polyethylene glycol(15) cetyl ether (ceteth-15), polyethylene glycol(16) cetyl ether (ceteth-16), polyethylene glycol(17) cetyl ether (ceteth-17), polyethylene glycol(18) cetyl ether (ceteth-18), polyethylene glycol(19) cetyl ether (ceteth-19), polyethylene glycol-(20) cetyl ether (ceteth-20),
polyethylene glycol(13) isocetyl ether (isoceteth-13), polyethylene glycol(14) isocetyl ether (isoceteth-14), polyethylene glycol(15) isocetyl ether (isoceteth-15), polyethylene glycol(16) isocetyl ether (isoceteth-16), polyethylene glycol(17) isocetyl ether (isoceteth-17), poly-ethylene glycol(18) isocetyl ether (isoceteth-18), polyethylene glycol(19) isocetyl ether (iso-ceteth-19), polyethylene glycol(20) isocetyl ether (isoceteth-20),
polyethylene glycol(12) oleyl ether (oleth-12), polyethylene glycol(13) oleyl ether (oleth-13), polyethylene glycol(14) oleyl ether (oleth-14), polyethylene glycol(15) oleyl ether (oleth-15),
polyethylene glycol(12) lauryl ether (laureth-12), polyethylene glycol(12) isolauryl ether (iso-laureth-12),
polyethylene glycol(13) cetylstearyl ether (ceteareth-13), polyethylene glycol(14) cetylstearyl ether (ceteareth-14), polyethylene glycol(15) cetylstearyl ether (ceteareth-15), polyethylene glycol(16) cetylstearyl ether (ceteareth-16), polyethylene glycol(17) cetylstearyl ether (ceteareth-17), polyethylene glycol(18) cetylstearyl ether (ceteareth-18), polyethylene glycol(19) cetylstearyl ether (ceteareth-19), polyethylene glycol(20) cetylstearyl ether (ceteareth-20).

It is also advantageous to choose the fatty acid ethoxylates from the following group:

polyethylene glycol(20) stearate, polyethylene glycol(21) stearate, polyethylene glycol(22) stearate, polyethylene glycol(23) stearate, polyethylene glycol(24) stearate, polyethylene glycol(25) stearate,
polyethylene glycol(12) isostearate, polyethylene glycol(13) isostearate, polyethylene-glycol(14) isostearate, polyethylene glycol(15) isostearate, polyethylene glycol(16) isostearate, polyethylene glycol(17) isostearate, polyethylene glycol(18) isostearate, poly-ethylene glycol(19) isostearate, polyethylene glycol(20) isostearate, polyethylene glycol(21) -isostearate, polyethylene glycol(22) isostearate, polyethylene glycol(23) isostearate, poly-ethylene glycol(24) isostearate, polyethylene glycol(25) isostearate,
polyethylene glycol(12) oleate, polyethylene glycol(13) oleate, polyethylene glycol(14) oleate, polyethylene glycol(15) oleate, polyethylene glycol(16) oleate, polyethylene glycol(17) oleate, polyethylene glycol(18) oleate, polyethylene glycol(19) oleate, polyethylene glycol(20) oleate.

The ethoxylated alkyl ether carboxylic acid or salt thereof which can be used is advantageously sodium laureth-11 carboxylate.

Sodium laureth-14 sulfate can be used advantageously as alkyl ether sulfate.

An advantageous ethoxylated cholesterol derivative which can be used is polyethylene glycol(30) cholesteryl ether. Polyethylene glycol(25) soyasterol has also proven successful.

Ethoxylated triglycerides which can be advantageously used are polyethylene glycol(60) evening primrose glycerides.

It is also advantageous to choose the polyethylene glycol glycerol fatty acid esters from the group polyethylene glycol(20) glyceryl laurate, polyethylene glycol(21) glyceryl laurate, poly-ethylene glycol(22) glyceryl laurate, polyethylene glycol(23) glyceryl laurate, polyethylene glycol(6) glyceryl caprate, polyethylene glycol(20) glyceryl oleate, polyethylene glycol(20) glyceryl isostearate, polyethylene glycol(18) glyceryl oleate/cocoate.

It is likewise favorable to choose the sorbitan esters from the group polyethylene glycol(20) sorbitan monolaurate, polyethylene glycol(20) sorbitan monostearate, polyethylene glycol-(20) sorbitan monoisostearate, polyethylene glycol(20) sorbitan monopalmitate, polyethylene glycol(20) sorbitan monooleate.

The coemulsifiers are advantageously chosen from the group of sorbitan esters and sucrose esters, in particular branched and unbranched alkyl esters and alkenyl esters having carbon chains of 4-24 carbon atoms, preferably sorbitan stearate, sorbitan oleate, glyceryl sorbitan stearate, sucrose monostearate, sucrose monolaurate, sucrose palmitate.

The coemulsifiers can advantageously be chosen from the group of monoglycerol monocarboxylic monoesters, in particular those characterized by the structures

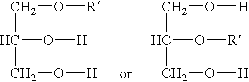

where R′ is a branched or unbranched acyl radical having 6-14 carbon atoms. R′ is advantageously chosen from the group of unbranched acyl radicals.

The acids on which these esters are based are

| | | |
|---|---|---|
| hexanoic acid | (caproic acid) | (R′ = —C₅H₁₁), |
| heptanoic acid | (enanthic acid) | (R′ = —C₆H₁₃), |
| octanoic acid | (caprylic acid) | (R′ = —C₇H₁₅), |
| nonanoic acid | (pelargonic acid) | (R′ = —C₈H₁₇), |
| decanoic acid | (capric acid) | (R′ = —C₉H₁₉), |
| undecanoic acid | | (R′ = —C₁₀H₂₁), |
| 10-undecenoic acid | (undecylenic acid) | (R′ = —C₁₀H₁₉), |
| dodecanoic acid | (lauric acid) | (R′ = —C₁₁H₂₃), |
| tridecanoic acid | | (R′ = —C₁₂H₂₅), |
| tetradecanoic acid | (myristic acid) | (R′ = —C₁₃H₂₇). |

R′ particularly advantageously represents the octanoyl radical (caprylic acid radical) or the decanoyl radical (capric acid radical), and is therefore represented by the formulae

R′═—C₇H₁₅ or R′=—C₉H₁₉.

The emulsifiers of the A type can also be advantageously chosen from the group of di- and triglycerol monocarboxylic monoesters. According to the invention, the di- or triglycerol units of the diglycerol monocarboxylic monoesters or friglycerol monocarboxylic monoesters according to the invention are in the form of linear, unbranched molecules, i.e. “monoglycerol molecules” etherified via the respective OH groups in the 1- or 3-position.

A low proportion of cyclic di- or triglycerol units, and glycerol molecules etherified via the OH groups in the 2-position, can be tolerated. It is, however, advantageous to keep such impurities as low as possible.

The monocarboxylic monoesters according to the invention are preferably characterized by the following structure:

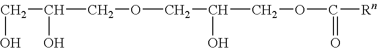

where R″ is a hydrocarbon radical, advantageously a branched or unbranched alkyl or alkenyl radical having 5 to 17 carbon atoms.

The monocarboxylic esters of triglycerol according to the invention are preferably characterized by the following structure:

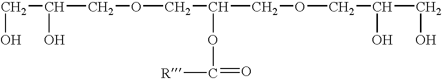

where R′″ is a hydrocarbon radical, advantageously a branched or unbranched alkyl or alkenyl radical having 5 to 17 carbon atoms.

The acids on which these esters are based are

| | | |
|---|---|---|
| hexanoic acid | (caproic acid) | (R″ and R″′ = —C₅H₁₁), |
| heptanoic acid | (enanthic acid) | (R″ and R″′ = —C₆H₁₃), |
| octanoic acid | (caprylic acid) | (R″ and R″′ = —C₇H₁₅), |
| nonanoic acid | (pelargonic acid) | (R″ and R″′ = —C₈H₁₇), |
| decanoic acid | (capric acid) | (R″ and R″′ = —C₉H₁₉), |
| undecanoic acid | | (R″ and R″′ = —C₁₀H₂₁), |
| 10-undecenoic acid | (undecylenic acid) | (R″ and R″′ = —C₁₀H₁₉), |
| dodecanoic acid | (lauric acid) | (R″ and R″′ = —C₁₁H₂₃), |
| tridecanoic acid | | (R″ and R″′ = —C₁₂H₂₅), |
| tetradecanoic acid | (myristic acid) | (R″ and R″′ = —C₁₃H₂₇), |
| pentadecanoic acid | | (R″ and R″′ = —C₁₄H₂₉), |
| hexadecanoic acid | (palmitic acid) | (R″ and R″′ = —C₁₅H₃₁), |
| heptadecanoic acid | (margaric acid) | (R″ and R″′ = —C₁₆H₃₃), |
| octadecanoic acid | (stearic acid) | (R″ and R″′ = —C₁₇H₃₅). |

R″ and R′″ are particularly favorably chosen from the group of unbranched alkyl radicals having an uneven number of carbon atoms, in particular 9, 11 and 13 carbon atoms.

In general, the monocarboxylic monoesters of diglycerol are preferably those of triglycerol.

According to the invention, very particular preference is given to

| | | |
|---|---|---|
| diglycerol monocaprate | (DMC) | R″ = 9 |
| triglycerol monolaurate | (TML) | R″′ = 11 |
| diglycerol monolaurate | (DML) | R″ = 11 |
| triglycerol monomyristate | (TMM) | R″′ = 13 |

A preferred monocarboxylic monoester of diglycerol according to the invention which has proven successful is diglycerol monocaprate (DMC).

In an advantageous embodiment of the present invention, an additional content of di- or triglycerol esterified in different positions is used, as is, where appropriate, a content of the various diesters of di- or triglycerol.

Also advantageous are triglyceryl diisostearate (nomenclature according to CTFA: polyglyceryl-3 diisostearate), isostearyidiglyceryl succinate, diglyceryl sesquiisostearate (nomenclature according to CTFA: polyglyceryl-2-sesquiisostearate), triglyceryl polyhydroxy-stearate (nomenclature according to CTFA: polyglyceryl-2 polyhydroxystearate).

Cetylstearyl isononanoate, dicocoylpentaerythrityldistearyl citrate, and also the methicone copolyols, cyclomethicone copolyols, alkylmethicone copolyols, in particular laurylmethicone copolyol, cetyldimethicone copolyol, have also proven advantageous according to the invention.

The coemulsifier(s) is/are particularly advantageously chosen from the group of branched or unbranched alkylmonocarboxylic acids, alkenylmonocarboxylic acids and alkylenedicarboxylic acids having 4 to 30 carbon atoms, in particular stearic acid, oleic acid, succinic acid, hexanoic acid (caproic acid), heptanoic acid (enanthic acid), octanoic acid (caprylic acid), nonanoic acid (pelargonic acid), decanoic acid (capric acid), undecanoic acid, undecenoic acid (undecylenic acid), dodecanoic acid (lauric acid), tridecanoic acid, tetra-decanoic acid (myristic acid), pentadecanoic acid, hexadecanoic acid (palmitic acid), hepta decanoic acid (margaric acid), octadecanoic acid (stearic acid), isostearic acid, behenic acid. It is also advantageous to choose the emulsifiers A from the group of cosmetically or pharmaceutically acceptable salts of the abovementioned carboxylic acids, in particular the alkali metal, ammonium, monoalkylammonium, dialkylammonium, trialkylammonium and tetraalkylammonium salts.

Advantageous coemulsifiers which can be chosen according to the invention are silicone emulsifiers, particularly advantageously those from the group of surface-active substances from the group of alkylmethicone copolyols and/or alkyldimethicone copolyols, preferably from the group of compounds characterized by the following chemical structure:

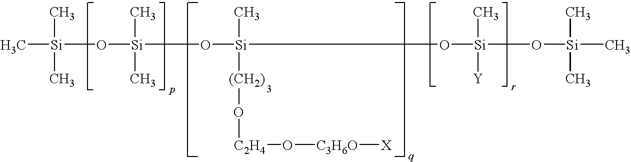

in which X and Y, independently of one another, are chosen from the group H and the branched and unbranched alkyl groups, acyl groups and alkoxy groups having 1-24 carbon atoms, p is a number from 0 to 200, q is a number from 1 to 40, and r is a number from 1 to 100.

One example of silicone emulsifiers to be used particularly advantageously for the purposes of the present invention are dimethicone copolyols which are sold by Th.Goldschmidt AG under the trade names ABIL® B 8842, ABIL® B 8843, ABIL® B 8847, ABIL® B 8851, ABIL® B 8852, ABIL® B 8863, ABIL® B 8873 and ABIL® B 88183.

A further example of surface-active substances to be used particularly advantageously for the purposes of the present invention is cetyldimethicone copolyol, which is sold by Th.Goldschmidt AG under the trade name ABIL® EM 90.

A further example of surface-active substances to be used particularly advantageously for the purposes of the present invention is cyclomethicone dimethicone copolyol, which is sold by Th.Goldschmidt AG under the trade name ABIL® EM 97.

Furthermore, the emulsifier laurylmethicone copolyol has proven very particularly advantageous, and is available under the trade name Dow Corning® 5200 Formulation Aid from Dow Corning Ltd.

The total amount of silicone emulsifiers used according to the invention in the cosmetic or dermatological preparations according to the invention is advantageously chosen from the range 0.1-10.0% by weight, preferably 0.5-5.0% by weight, based on the total weight of the preparations.

According to the invention, it is possible to multiply the use amounts of UV filters which are themselves sparingly soluble or insoluble in oil components, in particular tris(2-ethylhexyl) 4,4′,4″-(1,3,5-triazine-2,4,6-triyltriimino)trisbenzoate, but also 2-phenylbenzimidazole-5-sulfonic acid or salts thereof in cosmetic or dermatological preparations compared with the prior art.

The total amount of UV filter substances which are themselves sparingly soluble in oil components, in particular tris(2-ethylhexyl) 4,4′,4″-(1,3,5-triazine-2,4,6-triyltriimino)tris-benzoate, but also 2-phenylbenzimidazole-5-sulfonic acid and salts thereof in the finished cosmetic or dermatological preparations is advantageously chosen from the range 0.1-10.0% by weight, preferably 0.5-6.0% by weight, based on the total weight of the preparations.

It is advantageous according to the invention to use additional oil-soluble UVA filters and/or UVB filters in the lipid phase and/or water-soluble UVA filters and/or UVB filters in the aqueous phase in the preparations according to the invention.

The light protection formulations according to the invention can advantageously comprise further substances which absorb UV radiation in the UVB region, the total amount of filter substances being, for example, 0.1% by weight to 30% by weight, preferably 0.5 to 10% by weight, in particular 1 to 6% by weight, based on the total weight of the preparations, in order to make available cosmetic preparations which protect the skin from the entire range of ultraviolet radiation.

The additional UVB filters can be oil-soluble or water-soluble. Advantageous oil-soluble UVB filter substances are e.g.:
3-benzylidenecamphor derivatives, preferably 3-(4-methylbenzylidene)camphor, 3-benzylidenecamphor;
4-aminobenzoic acid derivatives, preferably 2-ethylhexyl 4-(dimethylamino)benzoate, amyl 4-(dimethylamino)benzoate;
esters of cinnamic acid, preferably 2-ethylhexyl 4-methoxycinnamate, isopentyl 4-methoxycinnamate;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably di(2-ethylhexyl) 4-methoxybenzalmalonate;
tris(2-ethylhexyl) 4,4′,4″-(1,3,5-triazine-2,4,6-triyltriimino)trisbenzoate.

Advantageous water-soluble UVB filter substances are e.g.:
salts of 2-phenylbenzimidazole-5-sulfonic acid, such as its sodium, potassium or its triethanolammonium salt, and the sulfonic acid itself,
sulfonic acid derivatives of benzophenones, preferably 2-hydroxy-4-methoxybenzo-phenone-5-sulfonic acid and salts thereof,
sulfonic acid derivatives of 3-benzylidenecamphor, such as, for example, 4-(2-oxo-3-bornylidenemethyl)benzenesulfonic acid, 2-methyl-5-(2-oxo-3-bornylidenemethyl)-sulfonic acid and salts thereof.

The list of said UVB filters which can be used in combination with the active ingredient combinations according to the invention is not of course intended to be limiting.

It can also be advantageous to use additional UVA filters in the preparations according to the invention which have hitherto been customarily present in cosmetic preparations. These substances are preferably derivatives of dibenzoylmethane, in particular 1-(4′-tert-butylphenyl)-3-(4′-methoxyphenyl)propane-1,3-dione and 1-phenyl-3-(4′-isopropylphenyl)-propane-1,3-dione. These combinations, and preparations which comprise these combinations, are also provided by the invention. The amounts which can be used are those used for the UVB combination.

The cosmetic and/or dermatological light protection formulations according to the invention can have the customary composition and be used for cosmetic and/or dermatological light protection, and also for the treatment, care and cleansing of skin and/or hair and as a make-up product in decorative cosmetics.

For use, the cosmetic and dermatological preparations according to the invention are applied to the skin and/or hair in sufficient amount and in the manner conventional for cosmetics.

Particularly preferred cosmetic and dermatological preparations are those which are in the form of a sunscreen. Advantageously, these can additionally contain at least one further UVA filter and/or at least one further UVB filter and/or at least one inorganic pigment, preferably an inorganic micropigment.

The cosmetic and dermatological preparations according to the invention can comprise cosmetic auxiliaries such as those conventionally used in such preparations, e.g. preservatives, bactericides, perfumes, antifoams, dyes, pigments which have a coloring effect, thickeners, moisturizers and/or humectants, fats, oils, waxes or other conventional constituents of a cosmetic or dermatological formulation, such as alcohols, polyols, polymers, foam stabilizers, electrolytes, organic solvents or silicone derivatives.

An additional content of antioxidants is generally preferred. According to the invention, favorable antioxidants which can be used are any antioxidants suitable or conventional for cosmetic and/or dermatological applications.

It is also advantageous to add antioxidants to the preparations according to the invention. The antioxidants are advantageously selected from the group consisting of amino acids (e.g. glycine, histidine, tyrosine, tryptophan) and derivatives thereof, imidazoles (e.g. urocanic acid) and derivatives thereof, peptides, such as D,L-carnosine, D-carnosine, L-carnosine and derivatives thereof (e.g. anserine), carotenoids, carotenes (e.g. α-carotene, β-carotene, ψ-lycopene) and derivatives thereof, chlorogenic acid and derivatives thereof, lipoic acid and derivatives thereof (e.g. dihydrolipoic acid), aurothioglucose, propylthiouracil and other thiols (e.g. thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts) and sulfoximine compounds (e.g. buthionine sulfoximine, homocysteine sulfoximine, buthionine sulfones, penta-, hexa-, heptathionine sulfoximine) in very low tolerated doses (e.g. pmol to μmol/kg), and also (metal) chelating agents (e.g. α-hydroxy fatty acids, palmitic acid, phytic acid, lactoferrin), α-hydroxy acids (e.g. citric acid, lactic acid, malic acid), humic acid, bile acid, bile extracts, bilirubin, biliverdin, EDTA, EGTA and derivatives thereof, unsaturated fatty acids and derivatives thereof (e.g. γ-linolenic acid, linoleic acid, oleic acid), folic acid and derivatives thereof, ubiquinone and ubiquinol and derivatives thereof, vitamin C and derivatives (e.g. ascorbyl palmitate, Mg ascorbyl phosphate, ascorbyl acetate), tocopherols and derivatives (e.g. vitamin E acetate), vitamin A and derivatives (vitamin A palmitate) and coniferyl benzoate of benzoin, rutinic acid and derivatives thereof, α-glycosylrutin, ferulic acid, furfurylideneglucitol, carnosine, butylhydroxytoluene, butyl-hydroxyanisole, nordihydroguaiacic acid, nordihydroguaiaretic acid, trihydroxy-butyrophenone, uric acid and derivatives thereof, mannose and derivatives thereof, zinc and derivatives thereof (e.g. ZnO, ZnSO₄), selenium and derivatives thereof (e.g. selenomethionine), stilbenes and derivatives thereof (e.g. stilbene oxide, trans-stilbene oxide) and the derivatives (salts, esters, ethers, sugars, nucleotides, nucleosides, peptides and lipids) of said active substances which are suitable according to the invention.

The amount of the abovementioned antioxidants (one or more compounds) in the preparations is preferably from 0.001 to 30% by weight, particularly preferably from 0.05 to 20% by weight, especially 1-10% by weight, based on the total weight of the preparation.

If vitamin E and/or derivatives thereof are used as the antioxidant or antioxidants, their respective concentrations are advantageously chosen from the range of 0.001-10% by weight, based on the total weight of the formulation.

If vitamin A or vitamin A derivatives or carotenes or derivatives thereof are used as the antioxidant or antioxidants, their respective concentrations are advantageously chosen from the range of 0.001-10% by weight, based on the total weight of the formulation.

The lipid phase can advantageously be chosen from the following group of substances:
mineral oils, mineral waxes
oils, such as triglycerides of capric or caprylic acid, but preferably castor oil;
fats, waxes and other natural and synthetic fatty substances, preferably esters of fatty acids with alcohols of low carbon number, e.g. with isopropanol, propylene glycol or glycerol, or esters of fatty alcohols with alkanoic acids of low carbon number or with fatty acids;
alkyl benzoates;
silicone oils such as dimethylpolysiloxanes, diethylpolysiloxanes, diphenyl-polysiloxanes and mixtures thereof.

For the purposes of the present invention, the oil phase of the emulsions, oleogels and hydrodispersions or lipodispersions is advantageously chosen from the group of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, from the group consisting of esters of aromatic carboxylic acids and saturated and/or unsaturated, branched and/or unbranched alcohols having a chain length of from 3 to 30 carbon atoms. Such ester oils can advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semisynthetic and natural mixtures of such esters, e.g. jojoba oil.

The oil phase can also advantageously be chosen from the group of branched and unbranched hydrocarbons and hydrocarbon waxes, silicone oils, dialkyl ethers, from the group of saturated or unsaturated, branched or unbranched alcohols, and also fatty acid triglycerides, namely the triglycerol esters of saturated and/or unsaturated, branched and/or unbranched alkanecarboxylic acids of chain length from 8 to 24, in particular 12-18, carbon atoms. The fatty acid triglycerides can advantageously be chosen, for example, from the group of synthetic, semisynthetic and natural oils, e.g. olive oil, sunflower oil, soybean oil, peanut oil, rapeseed oil, almond oil, palm oil, coconut oil, palm kernel oil and the like.

For the purposes of the present invention, any mixtures of such oil and wax components can also advantageously be used. When required, it can also be advantageous to use waxes, for example cetyl palmitate, as the sole lipid component of the oil phase.

The oil phase is advantageously chosen from the group consisting of 2-ethylhexyl isostearate, octyldodecanol, isotridecyl isononanoate, isoeicosane, 2-ethylhexyl cocoate, C₁₂₋₁₅-alkyl benzoate, caprylic/capric acid triglyceride and dicaprylyl ether.

Mixtures of C₁₂₋₁₅-alkyl benzoate and 2-ethylhexyl isostearate, mixtures of C₁₂₋₁₅-alkyl benzoate and isotridecyl isononanoate and mixtures of C₁₂₋₁₅-alkyl benzoate, 2-ethylhexyl isostearate and isotridecyl isononanoate are particularly advantageous.

Of the hydrocarbons, paraffin oil, squalane and squalene are advantageously to be used for the purposes of the present invention.

The oil phase can advantageously also contain cyclic or linear silicone oils or can consist entirely of such oils, although it is preferable to use an additional content of other oil phase components in addition to the silicone oil or silicone oils.

Cyclomethicone (octamethylcyclotetrasiloxane) is advantageously the silicone oil to be used according to the invention. However, other silicone oils can advantageously be used for the purposes of the present invention, for example hexamethylcyclotrisiloxane, polydimethylsiloxane, poly(methylphenylsiloxane).

Mixtures of cyclomethicone and isotridecyl isononanoate and mixtures of cyclomethicone and 2-ethylhexyl isostearate are particularly advantageous.

The aqueous phase of the preparations according to the invention may advantageously comprise
alcohols, diols or polyols of low carbon number, and also their ethers, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, and also alcohols having a low number of carbon atoms, e.g. ethanol, isopropanol, 1,2-propanediol, glycerol, and especially one or more thickeners which can advantageously be selected from the group consisting of silicon dioxide, aluminum silicates and polysaccharides and derivatives thereof, e.g. hyaluronic acid, xanthan gum, hydroxypropylmethylcellulose, and particularly advantageously from the group of polyacrylates, preferably a polyacrylate from the group consisting of the so-called Carbopols, for example Carbopol grades 980, 981, 1382, 2984, 5984, in each case individually or in combination.

The text below briefly discusses some peculiarities and differences in the prerequisites of O/W emulsions and O/W microemulsions according to the invention.

Oils and fats differ inter alia in their polarity, which is difficult to define. It has already been proposed to adopt the interfacial tension with respect to water as a measure of the polarity index of an oil or an oil phase. In this case, the lower the interfacial tension between this oil phase and water, the greater the polarity of the oil phase in question. According to the invention, the interfacial tension is to be regarded as one possible measure of the polarity of a given oil component.

The interfacial tension is the force which acts on an imaginary line one meter in length located in the interface between two phases. The physical unit of this interfacial tension is conventionally calculated from the force/length relationship and is usually expressed in mN/m (millinewtons divided by meters). It has a positive sign if it endeavors to reduce the interface. In the converse case, it has a negative sign.

According to the invention, the limit below which an oil phase is “polar” and above which an oil phase is “nonpolar” is regarded as 30 mN/m.

According to the invention, the oil phase is advantageously chosen for O/W microemulsions from the group of polar oil components which have a polarity between 10 and 30 mN/m, where it must be ensured that at least one nonpolar oil component is present.

Advantageous O/W microemulsions are obtained if the oil phase is chosen from the group of polar oil components, particularly preferably from the group of natural, synthetic or semisynthetic oil components, which have a polarity between 10 and 20 mN/m, where it must be ensured that at least one nonpolar oil component is present.

It is also advantageous to use polar vegetable oils as polar oils of the O/w emulsions according to the invention. The vegetable oils can advantageously be chosen from the group of oils from the plant families Euphorbiaceae, Poaceae, Fabaceae, Brassicaceae, Pedalaceae, Asteraceae, Linaceae, Flacourticaceae, Violales, preferably chosen from the group consisting of natural castor oil, wheatgerm oil, grapeseed oil, kukui nut oil, safflower oil, thistle, oil of evening primrose and further oils which comprise at least 1.5% by weight of linoleic acid glycerides.

The addition of electrolytes brings about a change in the solubility properties of a hydrophilic emulsifier. The hydrophilic emulsifiers having the structures or properties described above pass through a partial phase inversion, leading to solubilization of water by the oil phase, which results in a stable microemulsion.

The microemulsions according to the invention therefore advantageously comprise electrolytes, in particular one or more salts containing the following anions: chlorides, and also inorganic oxo element anions, and of these in particular sulfates, carbonates, phosphates, borates and aluminates. Electrolytes based on organic anions can also advantageously be used, for example lactates, acetates, benzoates, propionates, tartrates, citrates and many others. Comparable effects can also be achieved by ethylenediamine-tetraacetic acid and salts thereof.

Cations of the salts which are preferably used are ammonium, alkylammonium, alkali metal, alkaline earth metal, magnesium, iron and zinc ions. It goes without saying that only physiologically acceptable electrolytes are to be used in cosmetics. On the other hand, specific medicinal applications of the microemulsions according to the invention may, at least in principle, require the use of electrolytes which should not be used without medical supervision.

Particular preference is given to potassium chloride, sodium chloride, magnesium sulfate, zinc sulfate and mixtures thereof. Also advantageous are salt mixtures as occur in the natural salt from the Dead Sea.

The concentration of the electrolyte or of the electrolytes should be about 0.01-10.0% by weight, particularly advantageously about 0.03-8.0% by weight, based on the total weight of the preparation.

The emulsifiers of type A can be commonly regarded as O/W emulsifiers. A content of about 5-10% by weight of customary W/O emulsifiers advantageously promotes the formation of O/W/O emulsions, and a content of significantly more than 10% by weight of such emulsifiers leads to destabilization of the O/W/O emulsions.

If desired, for the preparation of O/W/O emulsions according to the invention, it is also advantageous to use hydrophilic and/or lipophilic gel formers. Although these do not generally contribute to the formation of multiple droplets, they promote the stability of multiple droplets once they have formed.

If, in a preparation process for O/W/O emulsions according to the invention, the pH is to be varied in order to bring an otherwise predetermined system into the phase inversion range, then it is advantageous to initially use as low an electrolyte concentration as possible in the water phase at the start of the process, and if possible to initially dispense with such a concentration entirely. It is also advantageous to introduce emulsifier A into the oil phase, for example for stearic acid in the concentration range 0.5-5% by weight, in particular 2% by weight. The presence of an emulsifier which is not covered by the definition of emulsifier A is advantageous in the concentration range from about 5-10% by weight, in particular about 7% by weight.

The pH should advantageously only be varied once the W/O emulsion has formed, for example by the addition of NaOH.

In this respect, it is within the general knowledge of the person skilled in the art and requires no inventive activity to determine the temperature and pH range in which phase inversion takes place for a given emulsifier or a given emulsifier system in a given water/oil phase system. As a general guideline for the PIT at customary emulsifier concentrations, a temperature range of about 40-90° C. can be stated. In general, the PIT decreases as the emulsifier concentration increases.

If desired, during this process, the basic substances, auxiliaries, additives and/or active ingredients customary in cosmetics or medicinal-pharmaceutical can also be added. It is clear to the person skilled in the art at which point in time such substances can be added to the process without the properties of the emulsion to be achieved being considerably impaired.

The examples below serve to outline the essence of the present invention in more detail without limiting the invention.

### EXAMPLE 1

### Self-Tanning Spray with Intensive Tanning Performance

| | |
|---|---|
| Cetylstearyl isononanoate | 8.00 |
| Ceteareth-20 | 6.00 |
| Dihydroxyacetone | 5.00 |
| Glycerol | 5.00 |
| Dicaprylyl ether | 4.00 |
| Vitamin E acetate | 0.50 |
| Glyceryl stearate | 2.00 |
| Sodium citrate | 0.50 |
| Citric acid | 0.20 |
| Dyes, perfume, preservative | 0.50 |
| Perfume | q.s. |
| Water | Ad. 100.00 |

### EXAMPLE 2

### Self-Tanning Spray with Intensive Tanning Performance

| | |
|---|---|
| Cetylstearyl isononanoate | 4.0000 |
| Ceteareth-15 | 6.0000 |
| Dihydroxyacetone | 5.0000 |
| Glycerol | 5.0000 |
| Dicaprylyl ether | 5.0000 |
| Vitamin E acetate | 0.5000 |
| Stearic acid | 2.3000 |
| Sodium hydroxide | 0.1070 |
| Citric acid | 0.2000 |
| Dyes, perfume, preservative | q.s. |
| Water | Ad. 100 |

### EXAMPLE 3

### Self-Tanning Spray with Average Tanning Performance

| | |
|---|---|
| C12-15-Alkyl benzoate | 7.5000 |
| Glycerol | 5.0000 |
| Cetylstearyl isononanoate | 5.0000 |
| Dihydroxyacetone | 2.0000 |
| Sorbitan monoisostearate | 4.0000 |
| Butylene glycol dicaprylate/dicaprate | 2.5000 |
| Vitamin E acetate | 0.5000 |
| Cetylstearyl alcohol | 1.5000 |
| Dyes, perfume, preservative | q.s. |
| Water | Ad. 100 |

### EXAMPLE 4

### Self-Tanning Spray with Weak Tanning Performance

| | |
|---|---|
| Dihydroxyacetone | 1.0000 |
| Glycerol | 5.0000 |
| Dicaprylyl ether | 5.0000 |
| Isoceteth-20 | 4.8000 |
| Cetylstearyl alcohol | 2.4000 |
| DMDM hydantoin | 0.4000 |
| Dyes, perfume, preservative | q.s. |
| Water | Ad. 100 |

### EXAMPLE 5

### Self-Tanning Spray with Additional Antioxidant Protection

| | |
|---|---|
| Dihydroxyacetone | 5.0000 |
| C₁₂₋₁₅-Alkyl benzoate | 4.5000 |
| Ceteareth-12 | 4.0000 |
| Cetylstearyl isononanoate | 2.5000 |
| Glycerol | 2.0000 |
| Dimethicone | 1.5000 |
| Dicaprylyl ether | 1.0000 |
| Glyceryl isostearate | 2.0000 |
| Cetyl alcohol | 1.0000 |
| DMDM hydantoin | 0.2000 |
| Glucosylrutin | 0.5000 |
| Perfume | q.s. |
| Water | Ad. 100 |

### EXAMPLE 6

### Self-Tanning Spray with High UV Protection

| | |
|---|---|
| Dicaprylyl ether | 3.0000 |
| Glycerol | 5.0000 |
| Octyltriazone | 1.0000 |
| Dioctylbutamidotriazone | 2.0000 |
| Anisotriazine | 1.0000 |
| Bisimidazylate | 0.5000 |
| Titanium dioxide | 0.5000 |
| Dihydroxyacetone | 5.0000 |
| Ceteareth-20 | 4.0000 |
| Butylene glycol dicaprylate/dicaprate | 5.0000 |
| Vitamin E acetate | 0.5000 |
| Cetylstearyl alcohol | 1.5000 |
| Dyes, perfume, preservative | q.s. |
| Perfume | q.s. |
| Water | Ad. 100 |

### EXAMPLE 7

### Self-Tanning Spray with Low UV Protection

| | |
|---|---|
| Glycerol | 5.0000 |
| Anisotriazine | 1.0000 |
| Butylmethoxydibenzoylmethane | 0.5000 |
| Bisimidazylate | 0.5000 |
| Dihydroxyacetone | 5.0000 |
| Ceteareth-20 | 4.0000 |
| Butylene glycol dicaprylate/dicaprate | 5.0000 |
| Vitamin E acetate | 0.5000 |
| Cetylstearyl alcohol | 1.5000 |
| Dyes, perfume, preservative | q.s. |
| Water | Ad. 100 |

What is claimed is:

1. A process for preparing a self-tanning oil-in-water microemulsion, wherein the process comprises changing a pH of a mixture comprising an aqueous phase, an oil phase, from 0.5% to 6% by weight of dihydroxyacetone and from 0.1% to 5% by weight of one or more emulsifiers A whose lipophilicity is pH-dependent, based on a total weight of the microemulsion, to effect a phase inversion which results in a sprayable, self-tanning oil-in-water microemulsion having an oil droplet diameter in a range of from about 10⁻¹ μm to about 10⁻² μm.

2. The process of claim 1, wherein the microemulsion has a pH of from 4.5 to 5.5.

3. The process of claim 1, wherein the microemulsion is translucent.

4. The process of claim 1, wherein the microemulsion further comprises one or more X-ray-amorphous oxide pigments.

5. The process of claim 4, wherein the one or more X-ray-amorphous oxide pigments are present at a concentration of from 0.5% to 10% by weight.

6. The process of claim 4, wherein the one or more X-ray-amorphous oxide pigments are present at a concentration of from 1% to 5% by weight.

7. The process of claim 1, wherein the microemulsion further comprises one or more substances which are soluble or dispersible in the oil phase.

8. The process of claim 1, wherein the microemulsion further comprises one or more water-in-oil (W/O) emulsifiers.

9. The process of claim 1, wherein the one or more emulsifiers A comprise one or more emulsifiers selected from polyethylene glycol cetylstearyl ethers.

10. The process of claim 9, wherein the one or more emulsifiers A comprise ceteareth-20.

11. A process for preparing a self-tanning oil-in-water microemulsion, wherein the process comprises (i) changing a pH of a mixture comprising an aqueous phase, an oil phase, from 0.5% to 6% by weight of dihydroxyacetone and from 0.1% to 5% by weight of one or more emulsifiers A whose lipophilicity is pH-dependent, based on a total weight of the microemulsion, to effect a phase inversion which results in a translucent, self-tanning oil-in-water microemulsion having an oil droplet diameter in a range of from about 10⁻¹ μm to about 10⁻² μm and (ii) adjusting a pH of the microemulsion to a value of from 4.5 to 5.5.

12. The process of claim 11, wherein the microemulsion is sprayable.

13. The process of claim 11, wherein the microemulsion further comprises one or more substances which are soluble or dispersible in the aqueous phase.

14. The process of claim 13, wherein the microemulsion further comprises one or more substances which are soluble or dispersible in the oil phase.

15. The process of claim 11, wherein the microemulsion further comprises one or more water-in-oil (W/O) emulsifiers.

16. The process of claim 11, wherein the one or more emulsifiers A comprise one or more emulsifiers having an HLB value of from 14.5 to 15.5.

17. The process of claim 11, wherein the one or more emulsifiers A comprise one or more emulsifiers selected from polyethylene glycol cetylstearyl ethers.

18. The process of claim 11, wherein the microemulsion further comprises from 1% to 5% by weight of one or more X-ray-amorphous oxide pigments.

19. A method of tanning skin, wherein the method comprises topically applying a microemulsion obtained by the process of claim 1 to skin in an amount which is effective to tan skin.

20. A method of tanning skin, wherein the method comprises topically applying a microemulsion obtained by the process of claim 11 to skin in an amount which is effective to tan skin.
